(19) United States
(12) Patent Application Publication
(10) Pub. No.: US 2012/0039961 A1
(43) Pub. Date: Feb. 16, 2012

(54) IMPLANT AND THERAPEUTIC COMPOSITION FOR TREATING DAMAGE AND/OR DISEASES RELATING TO THE HUMAN ANIMAL MUSCULOSKELETAL SYSTEM

(75) Inventor: Jürgen Mollenhauer, Reutlingen (DE)

(73) Assignee: TETEC TISSUE ENGINEERING TECHNOLOGIES AG, Reutlingen (DE)

(21) Appl. No.: 13/264,783

(22) PCT Filed: Apr. 16, 2010
(86) PCT No.: PCT/EP2010/002329

§ 371 Date: Oct. 18, 2011

(30) Foreign Application Priority Data
Apr. 17, 2009 (DE) 10 2009 018 640.9

(51) Int. Cl. A61K 9/70; A61P 19/00; A61P 21/00; A61P 17/02; A61K 35/32; C12N 5/077

(52) U.S. Cl. 424/400; 424/548; 435/325; 435/395

(57) ABSTRACT

An implant includes a support material, cartilage cells and/or precursor cells thereof, and a cartilage-specific collagen type. A method for preparing the implant, to a therapeutic composition, includes cartilage cells and/or precursor cells thereof and a cartilage-specific collagen type. The implant and the therapeutic composition are suitable in particular for treating damage and/or diseases relating to the human and/or animal musculoskeletal system.

### RELATED APPLICATIONS

This is a §371 of International Application No. PCT/EP2010/002329, with an international filing date of Apr. 16, 2010 (WO 2010/118874 A2, published Oct. 21, 2010), which is based on German Patent Application No. 10 2009 018 640.9, filed Apr. 17, 2009, the subject matter of which is incorporated by reference.

### TECHNICAL FIELD

This disclosure relates to an implant and to a therapeutic composition for treating damage and/or diseases relating to the human and/or animal musculoskeletal system, and to a method of preparing the implant and the therapeutic composition.

### BACKGROUND

Articular cartilage is capable only to a limited extent of repairing articular surface damage or damage between the vertebral bodies of the vertebral column. A composition based on collagen, which composition is also suitable in principle for repairing cartilage tissue, is known from EP 0 747 067 A2.

A further fundamental concept for the surgical treatment of cartilage damage envisages the use of cartilage implants based on cartilage cells prepared in vitro. The cartilage implants should substantially comprise autologous cells to keep the risk of transplant rejections in the patients concerned as low as possible.

Since, with the aid of biopsies, usually only few autologous cells can be provided, the isolated cartilage cells are normally subjected to multiple passages. However, a problem here is that a progressive loss of the differentiated cellular phenotype is associated with increasing passage number. This means that the isolated cartilage cells diverge ever further from their natural original state with respect to their properties the more frequently they are subjected to a technical proliferation cycle. Furthermore, the risk of a mutation and thus in general of tumorigenesis rises with increasing passage number.

A further problem in the in vitro culturing of cartilage cells in cell culture dishes, cell culture flasks, or the like is that the cells are usually not surrounded by an extracellular matrix which might retain, for example, substances produced and secreted by the cartilage cells. Instead, the cell-excreted substances, for example, soluble procollagens, immature proteoglycans, glycoprotein subunits, tissue hormones, growth factors, and specific extracellular proteases, are scattered diffusely in the artificial environment. To compensate for this loss of substances or materials, the abovementioned substances and other substances have to be continuously reproduced by the cartilage cells and reexcreted. This means that the cartilage cells have a permanently elevated metabolic rate, and this means, in turn, elevated stress conditions for the cartilage cells. This can lead to impairment of cartilage implants which are based on such cartilage cells.

It could therefore be helpful to provide an implant which avoids disadvantages of the prior art and enables in particular new cartilage formation, i.e., chondrogenesis, which is improved and, in particular, faster compared to conventional cartilage implants.

### SUMMARY

I provide an implant including a support material, cartilage cells and/or precursor cells thereof, and a cartilage-specific collagen that treats damage and/or diseases relating to a human and/or animal musculoskeletal system.

I also provide a therapeutic composition including cartilage cells and/or precursor cells thereof and a cartilage-specific collagen that treats damage and/or diseases relating to a human and/or animal musculoskeletal system.

I further provide a method for preparing an implant including a) providing cartilage cells and/or precursor cells thereof and a cartilage-specific collagen, and b) loading a support material with the cartilage cells and/or precursor cells thereof and the cartilage-specific collagen.

I further yet provide a method for preparing a therapeutic composition for treating damage and/or diseases relating to a human and/or animal musculoskeletal system, wherein an aqueous suspension, containing cartilage cells and/or precursor cells thereof, and an aqueous solution, containing a cartilage-specific collagen, are mixed.

### DETAILED DESCRIPTION

The implant comprises a support material, cartilage cells and/or precursor cells thereof, and a cartilage-specific collagen type. The implant can in principle be used both in human medicine and in veterinary medicine. The use in human medicine, more particularly in the field of regenerative medicine, is preferred. For instance, the implant is especially suitable for treating damage and/or diseases relating to the human and/or animal musculoskeletal system.

I found that, surprisingly, cartilage cells or precursor cells thereof “capture” cartilage-specific collagen, preferably type VI collagen, which is provided to them in vitro and use it for constructing a kind of extracellular matrix. Owing to the construction of an extracellular matrix around the cartilage cells, the substances secreted by the cells can be retained better and, as a result, the cells' loss of substances, described in the opening, is reduced. This slows down the cellular metabolic rate and, as a result, the stress on the cartilage cells is altogether lowered. The lower cellular stress and the construction of an extracellular matrix achieve altogether improved cartilage-inducing, i.e., chondrogenic, properties for implants compared to conventional implants.

Preferably, the cartilage cells and/or precursor cells thereof are of autologous and/or allogeneic, preferably autologous, origin. The cartilage cells and/or precursor cells thereof can in principle be of animal origin and/or originate from suitable cell lines. The cartilage cells and/or precursor cells thereof can in principle be of xenogeneic origin. For example, the cartilage cells and/or precursor cells thereof can be cells from pigs, horses, cattle, dromedaries, dogs and/or cats. Preferably, the cartilage cells and/or precursor cells thereof are of human origin. Particular preference is given to the use of autologous human cartilage cells and/or corresponding precursor cells. The cartilage cells and/or precursor cells thereof can, by methods familiar to those skilled in the art, be isolated from human or animal cartilage tissues, more particularly articular cartilage tissues and/or intervertebral disk tissues, and, if necessary, cultured.

The cartilage cells may be selected from the group consisting of chondrocytes, chondroblasts, and mixtures thereof. Chondroblasts (“cartilage formers”) generally mean the precursor cells of chondrocytes. They originate from mesenchymal stem cells and are the active form of the cartilage cell, since they can synthesize all the components of the cartilage matrix. As soon as this synthesis function has stopped, they differentiate into chondrocytes, the actual cartilage cells. The chondrocytes themselves are smaller than the chondroblasts, spherically shaped, have a rounded nucleus, and contain much water, fat, and glycogen. Their number, location, and density are specific for each cartilage type. Chondrocytes in the immature state can still divide, and this can lead to the characteristic emergence of “isogenic groups.” Production of these isogenic groups is based on the division of chondrocytes which are already surrounded by a cartilage matrix and can therefore no longer move apart. In other words, the isogenic groups are chondrocyte complexes in which each complex has evolved from a single chondrocyte. As soon as the chondrocytes are differentiated, they lose their ability to divide. The precursor cells are normally stem cells, preferably mesenchymal stem cells. The stem cells can be of animal or human origin, with human stem cells being preferred.

In principle, the cartilage cells can be passaged two or more times. A passage is understood to mean a technical proliferation cycle to increase the number of cells, which can be obtained usually only at a low number from biopsy material. For instance, the cartilage cells can be secondary, tertiary, quaternary cells or the like. Preferably, the cartilage cells are, however, primary cartilage cells. Primary cartilage cells have the advantage that they are very close to natural cartilage cells with respect to morphological and biochemical properties.

The cartilage cells and/or precursor cells thereof are particularly preferably embedded in the implant by means of in vitro colonization or in vitro culturing.

The cartilage-specific collagen type can in principle be of recombinant or microbiological origin. Preferably, the cartilage-specific collagen type is of animal, more particularly bovine, porcine and/or equine, and/or human origin.

Further, the cartilage-specific collagen type may originate from a biological, more particularly human or animal, tissue. Preferably, the cartilage-specific collagen type originates from a tissue selected from the group consisting of cornea, placental tissue, aortic tissue, synovial tissue, and mixtures thereof.

The cartilage-specific collagen type is preferably selected from the group consisting of type II, VI, IX, XI collagen and combinations thereof. The use of type VI collagen, more particularly water-soluble type VI collagen, is particularly preferred. This collagen type is the major constituent of the “pericellular matrix,” which directly surrounds the cartilage cells. The pericellular matrix encloses, like a cage, one or more cartilage cells. The cage-like arrangement of pericellular matrix and cartilage cells is also referred to as a chondron or territorium. The pericellular matrix should therefore be distinguished again from the actual extracellular matrix. In natural cartilage tissue, the extracellular matrix surrounds the chondrons just mentioned and thus the pericellular matrix. Type VI collagen is a heterotrimer, consisting of the polypeptides α1 (VI), α2 (VI), and α3 (VI). The abovementioned polypeptides have at their ends, in each case, a globular domain. The globular domains are normally spaced apart from one another by a short triple helical segment. This produces an overall dumbbell-shaped structure of monomeric type VI collagen. The monomers can assemble, via a lateral connection, to form tetramers. The tetramers in turn can be assembled, via their ends, to form structures which are like a string of pearls and filamentary.

The implant can further comprise not only the cartilage-specific collagen type, but also a cartilage-atypical collagen type, for example, from the group consisting of type I, III collagen and combinations thereof.

Preferably, the implant additionally comprises active biological substances. The active substances can be selected from the group consisting of extracellular proteases, antibodies, receptor antagonists, receptor agonists, hormones, growth factors, differentiation factors, recruitment factors, adhesion factors, antibiotics, antimicrobial compounds, anti-inflammatory compounds, immunosuppressive compounds, and combinations thereof.

Suitable recruitment components are, for example, chemotactics or chemotaxins. Adhesion factors which can be used are, for example, compounds from the group consisting of cytotactin, laminin, fibronectin, type IV, V and VII collagen, synthetic peptides which represent partial sequences of various adhesins, transmembrane connecting proteins, such as integrin for example, and combinations thereof.

The growth factors are preferably chondrogens, i.e., cartilage growth-inducing growth factors. Suitable growth factors can be selected from the group consisting of TGFs (transforming growth factors), BMPs (bone morphogenetic proteins), MPSFs (morphogenetic protein stimulatory factors), heparin-binding growth factors, inhibins, growth differentiation factors, activins, and combinations thereof. Preferably, the growth factors are selected from the group consisting of TGF-β1, TFG-β2, TFG-β3, BMP-1, BMP-2, BMP-3, BMP-4, BMP-5, BMP-6, BMP-7, BMP-8, BMP-9, FGF (fibroblast growth factor), EGF (epidermal growth factor), PDGF (platelet-derived growth factor), IGF (insulin-like growth factor), inhibin A, inhibin B, GDF-1 (growth differentiating factor I), activin A, activin B, activin AB, and combinations thereof. The use of TGFs and/or BMPs is particularly preferred. The growth factors can be isolated from native or natural sources, for example from mammalian cells. It is likewise possible for the growth factors to be prepared synthetically, for example, with the aid of recombinant DNA techniques or by chemical methods.

Particularly preferably, the implant has chondrons and/or chondron-like precursors. As already described, these are cage-like structures which are formed by a pericellular matrix together with one or more cartilage cells.

The support material is preferably designed as a three-dimensional structure or a three-dimensional network or matrix, more particularly protein matrix. The support material may or may not be crosslinked. Preferably, the support material is crosslinked, more particularly chemically crosslinked. The crosslinking reagents which can be used are aldehydes, dialdehydes, diisocyanates, or carbodiimides.

The support material preferably comprises a material which is selected from the group consisting of proteins and salts thereof, polysaccharides and salts thereof, polyhydroxyalkanoates, calcium phosphates, animal membranes, composites thereof and combinations thereof. Particularly advantageous proteins which may be mentioned are, in particular, fibrous proteins and salts thereof, more particularly extracellular proteins and salts thereof, preferably selected from the group consisting of collagen and salts thereof, gelatin and salts thereof, elastin and salts thereof, reticulin and salts thereof, and combinations thereof. Preferred collagens are selected from the group consisting of type I, II, III collagen and combinations thereof. The use of type II collagen as support material is particularly preferred, since it is the main collagen constituent of the extracellular matrix of natural cartilage tissue. The polysaccharides can be selected from the group consisting of cellulose derivatives, chitosan, chitosan derivatives, glycosaminoglycans, salts thereof and combinations thereof. The cellulose derivatives are preferably alkylcelluloses, hydroxyalkylcelluloses, carboxyalkylcelluloses, salts thereof and/or combinations thereof. Examples of suitable cellulose derivatives can thus be selected from the group consisting of methylcellulose, hydroxymethylcellulose, hydroxyethylcellulose, hydroxylethylmethylcellulose, hydroxypropylcellulose, hydroxypropylmethylcellulose, hydroxybutylcellulose, carboxymethylcellulose, salts thereof and combinations thereof. Suitable glycosaminoglycans can be selected from the group consisting of hyaluronic acid, heparin, heparan sulfate, chondroitin 4-sulfate, chondroitin 6-sulfate, dermatan sulfate, keratan sulfate, salts thereof and combinations thereof. Possible polyhydroxyalkanoates which can be mentioned are, in particular, polyglycolide, polylactide, poly-3-hydroxybutyrate, poly-4-hydroxybutyrate, copolymers thereof and mixtures thereof. Preferred calcium phosphates can be selected from the group consisting of fluorapatites, hydroxyapatites, tricalcium phosphate, tetracalcium phosphate, and combinations thereof. The animal membranes are preferably pericardial, more particularly of bovine origin.

Optionally, the support material can also contain reinforcing agents, more particularly reinforcing fibers, preferably selected from the group consisting of polysaccharide fibers, protein fibers, silk, cotton fibers, polylactide fibers, gelatin fibers, and combinations thereof.

The support material is normally designed to be porous, preferably openly porous. Advantageously, the support material has an interconnecting porosity. Furthermore, the support material can have pores with a pore size between 50 and 500 μm, more particularly between 50 and 350 μm, preferably between 100 and 200 μm.

The support material may be constructed as a multilayer. Preferably, the support material has at least a first layer and at least a second layer. The at least first layer and the at least second layer are normally connected to one another along a common interface. The connection along the common interface may be based on chemical and/or physical bonds. For example, the at least first layer and the at least second layer can be connected to one another by covalent bonds, more particularly by means of a chemical crosslinker. Furthermore, the at least first layer and the at least second layer can be adhesively bonded to one another. In an advantageous variant, the connection between the at least first layer and the at least second layer is achieved by lyophilization, more particularly colyophilization, of the layers.

The at least first layer preferably has a dense, more particularly cell-impermeable, structure. It can be envisaged that the at least first layer has a structure which is permeable to nutrients, active biological substances, active medical or pharmaceutical substances and/or, in general, to low molecular weight compounds. For example, the at least first layer can have pores with a pore size of <2 μm, more particularly <1 μm.

The at least second layer preferably has a porous, more particularly openly porous, structure. It is particularly preferred for the at least second layer to have a cell-permeable or cell-infiltratable structure. Preferably, the at least second layer has pores with a pore size between 50 and 250 μm, more particularly between 130 and 200 μm.

Further, the at least first layer may have a membrane-like structure. More particularly, the at least first layer can be designed as a membrane body. The at least second layer preferably has a sponge-like structure. More particularly, the at least second layer can be designed as a sponge body. With regard to the materials which the at least first layer and/or the at least second layer can comprise, reference is made to all the support materials described so far. It is, however, preferred for the at least first layer to comprise a material which is selected from the group consisting of collagen and salts thereof, more particularly type I and/or III collagen and salts thereof, elastin and salts thereof, bioresorbable polymers, pericardium, composites, glycosaminoglycans and salts thereof, and combinations thereof. The at least second layer preferably comprises a material, more particularly a hydrophilic material, which is preferably selected from the group consisting of collagen and salts thereof, more particularly type I and/or III collagen and salts thereof, hyaluronic acid and salts thereof, alginates and salts thereof, chitosan and salts thereof, gelatin and salts thereof, processed materials, composites, fibrin and salts thereof, and combinations thereof. The material of the at least second layer may also be crosslinked, more particularly chemically crosslinked.

Particularly preferably, the at least first layer is a pericardial membrane, more particularly a cattle pericardial membrane, and the at least second layer is a sponge body, preferably based on type I and/or III collagen or on salts thereof.

The at least first layer and the at least second layer have different resorption times. More particularly, the at least first layer has a longer resorption time than the at least second layer. The resorption times are preferably in vivo measured resorption times.

Preferably, the implant is a cartilage implant, more particularly an autologous cartilage cell implant. Preferably, the implant is a cartilage cell implant which comprises autologous cartilage cells and a biphasic three-dimensional collagen-based support material. The first phase of the support material is preferably designed as a sponge body, preferably with pores which have a column-like arrangement and are preferably connected to one another. This enables, particularly advantageously, a uniform three-dimensional distribution of the cartilage cells in the sponge body. The second phase of the support material is preferably an enveloping and, in particular, tear-resistant membrane, more particularly a pericardial membrane. The membrane prevents, particularly advantageously and in vivo, migration of the cartilage cells out of the area of defect. In addition, the membrane enables simple surgical handling, such as, for example, safe and easy sewing of the implant. A corresponding autologous chondrocyte cell implant is sold commercially under the name NOVOCART 3D.

The implant is preferably used in regenerative medicine, more particularly in the field of tissue engineering. As already mentioned, the implant is suitable in particular for treating damage to and/or diseases of the human and/or animal musculoskeletal system. The damage can be injuries which are caused, in particular, by traumatic events, for example, traffic accidents or sports accidents. The damage which can be treated by the implant can, however, also be the result of a systemic disease of the human and/or animal musculoskeletal system, for example, arthritis. Preferably, the implant is used in autologous or allogeneic, particularly preferably in autologous, cartilage cell transplantation. A further area of application of the implant relates to its use in the transplantation of autologous and/or allogeneic mesenchymal stem cells, more particularly for cartilage, tendon, ligament and/or bone regeneration. Particular preference is given to the use of the implant for treating damage to and/or diseases of human and/or animal cartilage tissue, more particularly articular cartilage tissue and/or intervertebral disk tissue. A further possible use of the implant relates to wound healing on or in the human and/or animal body.

A further aspect relates to a therapeutic composition, comprising cartilage cells and/or precursor cells thereof and a cartilage-specific collagen type. The cartilage cells are preferably selected from the group consisting of chondrocytes, chondroblasts, and combinations thereof. The precursor cells are normally stem cells, preferably mesenchymal stem cells. The cartilage cells and/or precursor cells thereof can be of human and/or animal origin. Preferably, the cartilage cells and/or precursor cells thereof are human cells. Furthermore, the cartilage cells and/or precursor cells thereof can be of autologous or allogeneic origin. Preferably, the cartilage cells and/or precursor cells thereof are autologous cells. The cartilage-specific collagen type preferably originates from a biological, more particularly human or animal, tissue, preferably selected from the group consisting of cornea, placental tissue, aortic tissue, synovial tissue, and combinations thereof. The cartilage-specific collagen type is preferably a collagen which is selected from the group consisting of type II, VI, IX, XI collagen and combinations thereof. As already mentioned, the use of type VI collagen is particularly preferred. The therapeutic composition can be in the form of an aqueous liquid, more particularly an aqueous dispersion, aqueous suspension or aqueous solution, hydrogel, or aqueous paste. Preferably, the therapeutic composition is in the form of an aqueous suspension. The therapeutic composition is suitable in particular for in vitro culturing or in vitro colonization of a support material with cartilage cells and/or precursor cells thereof. Furthermore, the composition is preferably used in autologous or allogeneic, preferably autologous, cartilage cell transplantation. In addition, the composition can also be used in the transplantation of autologous and/or allogeneic, preferably autologous, mesenchymal stem cells, more particularly for cartilage, tendon, ligament and/or bone regeneration. Particular preference is given to the use of the composition for treating damage to and/or diseases of human and/or animal cartilage tissue, more particularly articular cartilage tissue and/or intervertebral disk tissue. With regard to further features and advantages of the therapeutic composition, reference is made in full to the description up to this point.

A further aspect comprises a preparation method for the implant, comprising the steps of:
a) providing cartilage cells and/or precursor cells thereof and a cartilage-specific collagen type, and
b) loading or inoculating a support material with the cartilage cells and/or precursor cells thereof and the cartilage-specific collagen type.

Preferably, the cartilage cells and/or precursor cells thereof together with the cartilage-specific collagen type are provided in the form of an aqueous mixture. The aqueous mixture, in turn, can be provided in the form of an aqueous dispersion, aqueous suspension, aqueous solution, hydrogel, or aqueous paste. To prepare the aqueous mixture, preferably an aqueous suspension of the cartilage cells and/or of the precursor cells thereof is used. For this purpose, the cartilage cells and/or precursor cells thereof can, if necessary, be pretreated enzymatically. The cartilage cells themselves can be taken from corresponding cell lines, cell cultures and/or biopsy samples. Furthermore, preferably a solution or suspension of the cartilage-specific collagen type is used to prepare the aqueous mixture. The solution or suspension preferably has a cartilage-specific collagen type concentration of between 10 μg/ml and 10 mg/ml. To prepare the aqueous mixture, more particularly the suspension, of the cartilage cells and/or of the precursor cells thereof and/or the solution or suspension of the cartilage-specific collagen type, all biofluids can be used in principle. For example, biofluids from the group consisting of water, buffer solutions, electrolyte solutions, nutrient solutions and body fluids, for example, blood and/or synovial fluid, can be used. The aqueous mixture can also contain active biological substances, more particularly at a concentration between 10 and 500 ng/ml.

The support material may initially be loaded only with the cartilage cells and/or precursor cells thereof. In this case, the cells are left to migrate into the support material, normally over a particular period which is typically in the range from 1 to 2 days, before the support material is subsequently loaded with the cartilage-specific collagen type.

Preferably, coincubation of the cartilage cells and/or of the precursor cells thereof and of the cartilage-specific collagen type, preferably in the aqueous mixture already mentioned, is carried out prior to loading the support material. The coincubation can be carried out within a period of between 1 and 48 hours, more particularly between 2 and 36 hours, preferably between 4 and 24 hours. Alternatively or in combination therewith, coincubation of the cartilage cells and/or of the precursor cells thereof and of the cartilage-specific collagen type can be carried out on or in the support material. The incubation time is, in this case, preferably between 2 and 24 hours. The coincubations described in this paragraph are preferably carried out in a temperature range between 0 and 39° C., more particularly between 30 and 39° C.

I also provide an implant which is prepared or can be prepared according to any of the methods described above.

A further aspect relates to a method of preparing the therapeutic composition. To prepare the composition, an aqueous liquid, preferably an aqueous suspension, containing cartilage cells and/or precursor cells thereof, and an aqueous solution (or aqueous suspension), containing a cartilage-specific collagen type, are mixed.

To prepare the aqueous suspension, the cartilage cells and/or precursor cells thereof can, if necessary, be pretreated enzymatically. The aqueous solution (or aqueous suspension) of the cartilage-specific collagen type preferably has a cartilage-specific collagen type concentration of between 10 μg/ml and 10 mg/ml. With regard to further features and properties, particularly with respect to the cartilage cells and/or precursor cells thereof and to the cartilage-specific collagen type, reference is made in full to the description up to this point.

Lastly, this disclosure relates to the use of a support material, of cartilage cells and/or precursor cells thereof, and of a cartilage-specific collagen type to prepare an implant, particularly for treating damage and/or diseases relating to the human and/or animal musculoskeletal system. Furthermore, the disclosure also relates to the use of an aqueous suspension, containing cartilage cells and/or precursor cells thereof, and an aqueous solution (or aqueous suspension), containing a cartilage-specific collagen type, for preparing a therapeutic composition, preferably for treating damage and/or diseases relating to the human and/or animal musculoskeletal system. With regard to further features and details, particularly with respect to the cartilage cells and/or the precursor cells thereof and to the cartilage-specific collagen type, reference is similarly made in full to the description up to this point.

Further features and advantages of the disclosure are evident from the following description in the form of examples. The features can be implemented each on their own or by way of combining a plurality thereof with one another. The following example serves merely for illustration and should in no way be considered to be limiting.

### Example

Articular cartilage was excoriated from the radial head of a 37-year-old female patient, freed from all blood and tissue residues, and chopped in a Petri dish using scalpels. The cartilage pieces were added to the sieve of a digestion chamber with a magnetic tube and stirred with 50 ml of hyaluronidase solution (25 mg of hyaluronidase in 50 ml of PBS (phosphate-buffered sodium chloride solution or phosphate-buffered saline)) at 37° C. for 25 minutes. Afterwards, the tissue was incubated with 50 ml of 0.25% by weight trypsin/EDTA (45 minutes, 37° C., stirred) and washed for 5 minutes with 50 ml of DMEM (Dulbecco's modified Eagle medium, high glucose) plus 10% by volume FCS (fetal calf serum). The cartilage cells were liberated from the tissue structure using a triple collagenase treatment, in which the tissue was incubated at 37° C. with stirring with 50 ml of collagenase solution (25 mg in 50 ml of DMEM plus 10% by volume FCS plus 100 U/ml penicillin plus 100 μg/ml streptomycin) and the cells were removed by centrifugation (500×g, 5 minutes) from the solution which had dripped through. The tissue was digested with collagenase initially for 2 hours, later twice overnight. Subsequently, further cells were rinsed from the tissue by addition of DMEM plus 10% by volume FCS and removed by centrifugation as before.

The isolated cartilage cells were subsequently made up as an aqueous suspension. The aqueous suspension was subsequently mixed with an aqueous solution, containing type VI collagen. In the mixture prepared, the cartilage cells and the type VI collagen were coincubated over a period of about 18 hours. Subsequently, a biocompatible support material (scaffold) was loaded with the mixture. The support material was a composite material based on a first layer designed to be membrane-like and on a second layer designed to be sponge-like. The first layer was a pericardial membrane. The second layer was substantially formed from crosslinked gelatin. After the composite material had been loaded, about 24 hours were allowed to elapse so that the second layer designed to be sponge-like could be infiltrated by the cartilage cells to a sufficient extent.

Subsequently, the thus loaded composite material was successfully implanted into a defective articular cartilage region in the abovementioned female patient.

1. An implant comprising a support material, cartilage cells and/or precursor cells thereof, and a cartilage-specific collagen that treats damage and/or diseases relating to a human and/or animal musculoskeletal system.

2. The implant as claimed in claim 1, wherein the cartilage cells and/or precursor cells thereof are of autologous or allogeneic, preferably autologous, origin.

3. The implant as claimed in claim 1, wherein the cartilage cells are primary cartilage cells.

4. The implant as claimed in claim 1, wherein the cartilage cells and/or precursor cells thereof are embedded in the implant by in vitro colonization or in vitro culturing.

5. The implant as claimed in claim 1, wherein the cartilage-specific collagen is of animal, more particularly bovine, porcine and/or equine, and/or human origin.

6. The implant as claimed in claim 1, wherein the cartilage-specific collagen originates from a tissue selected from the group consisting of cornea, placental tissue, aortic tissue, and synovial tissue.

7. The implant as claimed in claim 1, wherein the cartilage-specific collagen is selected from the group consisting of type II, VI, IX and XI collagen.

8. The implant as claimed in claim 1, wherein the cartilage-specific collagen is type VI collagen.

9. The implant as claimed in claim 1, wherein the implant has chondrons and/or chondron-like precursors.

10. The implant as claimed in claim 1, wherein the support material comprises a material selected from the group consisting of proteins and salts thereof, polysaccharides and salts thereof, polyhydroxyalkanoates, animal membranes, and combinations thereof.

11. The implant as claimed in claim 1, wherein the support material has a pore size between 50 and 500 μm.

12. The implant as claimed in claim 1, wherein the support material has at least a first layer and at least a second layer.

13. The implant as claimed in claim 12, wherein the at least first layer has a membrane structure and the at least second layer has a sponge structure.

14. The implant as claimed in claim 1, used in tissue engineering.

15. The implant as claimed in claim 1, used in wound healing.

16. The implant as claimed in claim 1, used in autologous or allogeneic cartilage cell transplantation.

17. The implant as claimed in claim 1, used in transplantation of autologous or allogeneic mesenchymal stem cells for cartilage, tendon, ligament and/or bone regeneration.

18. The implant as claimed in claim 1 for treating damage to and/or diseases of human and/or animal cartilage tissue.

19. A therapeutic composition comprising cartilage cells and/or precursor cells thereof and a cartilage-specific collagen that treats damage and/or diseases relating to human and/or animal musculoskeletal system.

20. A method for preparing an implant comprising:
a) providing cartilage cells and/or precursor cells thereof and a cartilage-specific collagen, and
b) loading a support material with said cartilage cells and/or precursor cells thereof and said cartilage-specific collagen.

21. The method as claimed in claim 20, wherein the cartilage cells and/or precursor cells thereof together with the cartilage-specific collagen are provided in the form of an aqueous mixture.

22. The method as claimed in claim 21, wherein an aqueous suspension of the cartilage cells and/or of the precursor cells thereof is used to prepare the aqueous mixture.

23. The method as claimed in claim 21, wherein a solution of the cartilage-specific collagen is used to prepare the aqueous mixture and the solution has a cartilage-specific collagen type concentration of 10 μg/ml to 10 mg/ml.

24. The method as claimed in claim 20, wherein coincubation of the cartilage cells and/or of the precursor cells thereof and of the cartilage-specific collagen type is carried out prior to loading the support material.

25. The method as claimed in claim 20, wherein coincubation of the cartilage cells and/or of the precursor cells thereof and of the cartilage-specific collagen type is carried out on or in the support material.

26. An implant prepared by a method as claimed in claim 20.

27. A method for preparing a therapeutic composition for treating damage and/or diseases relating to a human and/or animal musculoskeletal system, wherein an aqueous suspension, containing cartilage cells and/or precursor cells thereof, and an aqueous solution, containing a cartilage-specific collagen, are mixed.

28. (canceled)

29. (canceled)
